(19) United States
(12) Patent Application Publication
(10) Pub. No.: US 2024/0189186 A1
(43) Pub. Date: Jun. 13, 2024

(54) METHODS AND SYSTEMS FOR FILLING CLIMATE CONTROLLED MEDICATIONS

(71) Applicant: Express Scripts Strategic Development, Inc., St. Louis, MO (US)

(72) Inventor: Robert E. Hoffman, Linden, IN (US)

(21) Appl. No.: 18/536,519

(22) Filed: Dec. 12, 2023

Related U.S. Application Data
(60) Provisional application No. 63/431,824, filed on Dec. 12, 2022.

(51) Int. Cl. A61J 1/16; G16H 20/10

(52) U.S. Cl. CPC A61J 1/165; G16H 20/10; A61J 2200/50

(57) ABSTRACT

The temperature-controlled medication packaging system includes a storage with a temperature-controlled interior that contains at least one medication that is disposed within a shipping box. The temperature-controlled medication packaging system also includes at least one shipping container in a packager. The temperature-controlled medication packaging system further includes at least one coolant that is sized to fit within the at least one shipping container. At least one robot is adapted to retrieve the at least one medication directly from the shipping box within the storage and transport the at least one medication to the shipping container in the packager. The at least one robot is also adapted to retrieve the at least one coolant and to transport the at least one coolant to the shipping container in the packager.

### CROSS-REFERENCE TO RELATED APPLICATION

This application claims priority to U.S. Provisional Patent App. No. 63/431,824, filed on Dec. 12, 2022, and entitled “METHODS AND SYSTEMS FOR FILLING CLIMATE CONTROLLED MEDICATIONS,” the entire contents of which is herein incorporated by reference.

### FIELD

The present disclosure relates generally to the technical field of automated filling centers. In a specific example, the present disclosure can relate to a high-volume fulfillment center (e.g., a high-volume pharmacy, etc.) and to systems and methods for handling packaged units.

### BACKGROUND

The background description provided here is for the purpose of generally presenting the context of the disclosure. Work of the presently named inventors, to the extent it is described in this background section, as well as aspects of the description that cannot otherwise qualify as prior art at the time of filing, are neither expressly nor impliedly admitted as prior art against the present disclosure.

An automated pharmacy can process and fill a large number of prescriptions and prescription orders. Automated systems can be used by a high-volume pharmacy to process and fulfill prescriptions.

Mail order pharmacies provide a convenient and cost-effective option for patients to receive prescription drugs. For example, a mail order pharmacy can be capable of taking advantage of economies of scale, volume dispensing of prescription drugs, and centralized warehousing and shipping to reduce the cost of prescription drugs purchased by patients of the mail order pharmacy.

### SUMMARY OF THE INVENTION

A temperature-controlled medication packaging system that includes a storage with a temperature-controlled interior that contains at least one medication that is disposed within a shipping box. The temperature-controlled medication packaging system also includes at least one shipping container in a packager. The temperature-controlled medication packaging system further includes at least one coolant that is sized to fit within the at least one shipping container. At least one robot is adapted to retrieve the at least one medication directly from the shipping box within the storage and transport the at least one medication to the shipping container in the packager. The at least one robot is also adapted to retrieve the at least one coolant and to transport the at least one coolant to the shipping container in the packager.

According to another aspect of the present disclosure, the shipping box has an open top, and the at least one robot includes a first robot that is adapted to remove the at least one medication from the shipping box through the open top.

According to yet another aspect of the present disclosure, the storage is held at a temperature that is no greater than forty degrees Fahrenheit.

According to still another aspect of the present disclosure, the storage has a curtain, and the at least one robot includes a first robot that can penetrate through the curtain when retrieving the at least one medication directly from the shipping box.

According to a further aspect of the present disclosure, the storage includes a plurality of shipping boxes that are disposed on at least one platform, and the plurality of shipping boxes have open tops.

According to yet a further aspect of the present disclosure, the at least one platform includes a plurality of platforms that contain different types of medications, and all of the plurality of platforms are within a reach of the first robot.

According to still a further aspect of the present disclosure, the shipping container is an insulated bag that acts as a moisture barrier between an environment outside the shipping container and an inside of the shipping container that can contain the at least one medication and the at least one coolant.

According to another aspect of the present disclosure, the at least one robot includes a first robot that is adapted to retrieve the at least one medication directly from the shipping box and deliver the at least one medication to the shipping container in the package and includes a second robot that is adapted to retrieve and deliver to the shipping container at least one the at least one coolant and a filler and a temperature sensor and paperwork.

Another aspect of the present disclosure is related to a method of filling a shipping container with a medication. The method includes the step of picking at least one medication directly from a shipping box that is located within an environmentally controlled storage with at least one robot. The method continues with the step of delivering the at least one medication to a packager that includes at least one shipping container with at least one robot. The method proceeds with the step of picking at least one coolant from a coolant supply and delivering the at least one coolant to the packager with the at least one robot.

According to another aspect of the present disclosure, the method further includes the step of picking and delivering at least one of a filler, a temperature sensor, and paperwork to the packager with the at least one robot.

According to yet another aspect of the present disclosure, the at least one robot includes a first robot and a second robot. The first robot performs the steps of picking the at least one medication from the shipping container and delivering the at least one medication to the packager. The second robot performs the steps of picking and delivering the at least one of the filler, the temperature sensor, and the paperwork to the packager.

According to still another aspect of the present disclosure, the shipping box has an open top, and the at least one robot picks the at least one medication from the shipping box through the open top.

According to a further aspect of the present disclosure, the method further includes the step of maintaining the environmentally controlled storage at a temperature that is no greater than forty degrees Fahrenheit.

According to yet a further aspect of the present disclosure, the environmentally controlled storage includes a curtain, and during the step of picking the at least one medication directly from the shipping box, the at least one robot penetrates through the curtain.

According to still a further aspect of the present disclosure, the environmentally controlled storage includes a plurality of shipping boxes that are disposed on at least one platform, and the plurality of shipping boxes have open tops.

According to another aspect of the present disclosure, the at least one platform includes a plurality of platforms that contain different types of medications, and all of the plurality of platforms are within a reach of the at least one robot.

According to yet another aspect of the present disclosure, the shipping container is an insulated bag that acts as a moisture barrier between an environment outside the shipping container and an inside of the shipping container that can contain the at least one medication and the at least one coolant.

Yet another aspect of the present disclosure is related to a temperature-controlled medication packaging system that includes an environmentally controlled storage with a temperature-controlled interior that contains a plurality of medications that are disposed within a plurality of shipping boxes. The shipping boxes having openings. The temperature-controlled medication packaging system also includes a coolant supply including at least one coolant, a filler supply including a filler material, a paperwork producer including paperwork related to the medications, and a packager including at least one insulated shipping container. A first robotic arm has a range of motion that includes at least the coolant supply and the packager. A second robotic arm has a range of motion that includes the packager and also includes at least one of the coolant supply, the filler supply, and the paperwork producer. A controller is in electrical communication with the first and second robotic arms. The controller is configured to operate the first robotic arm to retrieve at least one medication of the plurality of medications directly from one of the shipping boxes and deliver the at least one medication to the packager. The controller is also configured to operate the second robotic arm to retrieve and deliver to the packager at least one of the at least one coolant, the filler material, and the paperwork.

According to another aspect of the present disclosure, the plurality of shipping boxes in the environmentally controlled storage are disposed on at least one platform. The plurality of shipping boxes have open tops.

According to yet another aspect of the present disclosure, the at least one platform includes a plurality of platforms that support shipping boxes that contain different types of medications.

### BRIEF DESCRIPTION OF THE DRAWINGS

The present disclosure will become more fully understood from the detailed description and the accompanying drawings.

FIG. 1 is a diagram of an example implementation of a system for an automated pharmacy, according to an example embodiment;

FIG. 2A is a perspective view of an example package with the package being held in an open condition;

FIG. 2B is another perspective view of the example package and showing an insulating layer;

FIG. 2C is a front elevation view of the example package;

FIG. 3 is an example process flow illustrating a method for filling a prescription order for an environmentally controlled drug in an automated pharmacy, according to an example embodiment; and

FIG. 4A is a diagram of an example implementation of a system for an automated pharmacy, according to an example embodiment.

FIG. 4B is a diagram of an example implementation of a system for an automated pharmacy, according to an example embodiment.

FIG. 5A is a diagram of an example implementation of a system for an automated pharmacy, according to an example embodiment;

FIG. 5B is a shipping box that contains a plurality of unit-of-use products;

FIG. 6 is a partial fragmentary view showing a package of the type that may be produced by the system of FIG. 1;

FIG. 7 is a schematic view showing an electrical system of the exemplary system of FIG. 1; and

FIG. 8 is a perspective view of a second exemplary embodiment of the system.

In the drawings, reference numbers can be reused to identify similar and/or identical elements.

### DETAILED DESCRIPTION

Example systems and methods for filling drug orders including an environmentally controlled drug, for example, in an automated pharmacy, are described. In the following description, for purposes of explanation, numerous specific details are set forth in order to provide a thorough understanding of example embodiments. It will be evident, however, to one of ordinary skill in the art that these embodiments can be practiced without these specific details.

Generally, a prescription order is generated for an automated pharmacy, which can be a specialty pharmacy or a high-volume pharmacy. A high-volume pharmacy dispenses prescription drugs in a high volume. A high-volume pharmacy can include automatic pill dispensing systems to carry out the dispensing of the prescription drugs automatically at a rapid rate. A specialty pharmacy focuses on high cost, high touch medication therapy for patients with complex disease states. Medications in a specialty pharmacy range from oral to cutting edge injectable and biologic products. In each of these pharmacies, a drug can require environmental control while in storage and during shipping. The drugs can also include pre-packaged drugs that are ready to be sent to the consumer. Such drug packaging may be referred to a unit-of-use. The prescription order can include more than one prescription drug for fulfillment. Each prescription drug in a prescription order is an order component of the prescription order. Generally, the order components are vials, pill bottles; liquid bottles; blister packs; nasal sprays; unit-of-use packs or products; injectable packages; spray bottles; tubes; ampoules; drop counters; insulated boxes; child-resistant containers; or other packaging having a quantity of a prescription drug contained therein. At least one of the drugs in an order can require a controlled environment. An example of a controlled environment is one that maintains a required temperature range and/or humidity range. The controlled environment can be a lowered temperature. By way of example, certain insulin needs to be stored at a temperature less than room temperature, for example at less than 60° F. (16° C.), less than 45° F. (7° C.), less than 40° F. (4° C.), or less than 32° F. (0° C.), inclusive of ranges therebetween.

The prescription drugs can be dispensed at various sections of the automated pharmacy, e.g., a high-volume pharmacy or a specialty pharmacy. Some prescription orders can require manual handling of certain order components. Some prescription order components can be filled automatically by filling machinery. It is one goal of the present disclosure to provide an automated system to fill prescription orders that include an environmentally controlled drug. The system can include at least two robots to retrieve, package, and prepare for shipping the prescription order with an environmentally controlled drug. The robots can operate autonomously, e.g., without intervention by a person.

One example of a drug that requires temperature control is HUMIRA® (adalimumab), which can be supplied as a unit-of-use product, e.g., in prefilled syringes as a preservative-free, sterile solution for subcutaneous administration. There are various unit-of-use products or packages of Humira®. For example, one unit-of-use product of Humira® is a package containing a pen carton containing two alcohol preps, two dose trays, six alcohol preps, and six dose trays (or other numbers of dose trays). Each dose tray consists of a single-use pen, containing a 1 mL prefilled glass syringe with a fixed 27-gauge ½ inch needle, providing 40 mg (0.8 mL) of HUMIRA®. Like many drugs, HUMIRA® must be refrigerated, e.g., at 36ºF to 46° F. (2° C. to 8° C.) and not be frozen, e.g., a temperature above 32ºF (0° C.).

Another example of a drug that may require environmental control is a nasal influenza (flu) vaccine. A nasal spray influenza (flu) vaccines can be a multirivalent vaccine, e.g., quadrivalent, meaning the vaccine is designed to protect against four flu viruses, sush as an influenza A(H1N1) virus, an influenza A(H3N2) virus and two influenza B viruses. An example is FluMist Quadrivalent manufactured by AstraZeneca Corporation. The FluMist should be stored between 35-46ºF (2-8° C.), above freezing but less than room temperature. The nasal vaccine may provide a dose in the form of a spray puff of drug suspended in a liquid.

A drug that requires temperature control cannot be used if it falls outside of its excursion time period. The excursion time period is the time a drug can be outside of its environmental control and remain a useable drug. The excursion time period can be a function of the time outside the environmental control and the magnitude of the change outside of the environmental control. In the case of a temperature-controlled drug, the excursion time period can be function of time and the temperature difference between the controlled environment and the non-controlled environment. In the case of drugs, the pharmacist can set the excursion time period. A temperature-controlled drug can have a storage temperature range for a specific time period, which can be zero seconds, less than one minute, less than two minutes, or less than five minutes. If a drug becomes frozen, then it typically cannot be thawed and used. When shipping or traveling with a temperature-controlled drug (for example, FluMist, HUMIRA® or the like), it must be stored in a shipping container that includes a cold pack, which could be in the form of an ice pack or another thermally controlled carrier, to maintain the drug in the predetermined temperature range until the drug reaches its destination. Additionally, some drugs must be protected from direct sunlight, during storage and transport.

Another drug that requires environmental control is ENBREL®, which is stored between 36° F. and 46° F. (2° C. and 8° C.). This is the standard temperature for many home refrigerators. However, you can keep ENBREL® at room temperature (between 68° F. and 77° F., or 20° C. and 25° C.) for up to 14 days. Storing ENBREL® at room temperature in an automated pharmacy is not standard practice.

Other temperature-controlled drugs (including both prescription and over the counter drugs) can require different temperature ranges than ENBREL® or HUMIRA®, and the present disclosure is not limited just to these two exemplary medications. The present system can store the drugs at their required temperature ranges by storing the drugs, for example, in different cooler packages or in different zones of a single cooler package. The refrigerated cooler that stores the medication at the pharmacy also stores the medications in the required temperature ranges. It is within the scope of the present disclosure to store drugs and order components (such as sharps packages and instructions) and fill to orders with those drugs and order components along with a coolant to maintain the drugs in their required temperature ranges during shipping using the system and methods described herein.

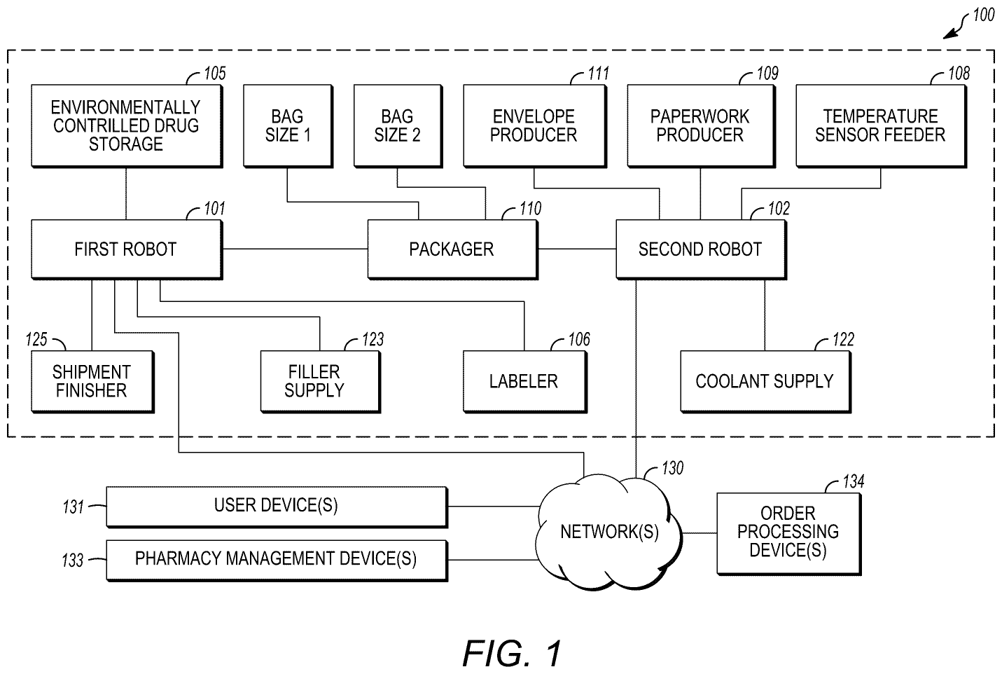

FIG. 1 shows a diagram of an example implementation of an automated pharmacy system 100 for filling a shipping container including a temperature-controlled drug, and FIG. 6 shows an exemplary shipping package 600 produced by the system 100. The system 100 can be part of an automated pharmacy, e.g., a high-volume pharmacy. The system 100 uses multiple packing robots 101, 102 that can essentially access at least some distinct parts of the system 100 with at least one point or area or station where both robots 101, 102 have access. The robots 101, 102 can have respective individual arcs of movement to travel between multiple stations and move a medication or other objects related to a pharmaceutical order between the stations. A station can be a location at which a function is performed to complete an order, e.g., in the pharmacy. In an example embodiment, the robots 101, 102 can deliver a unit-of-use product 602 and accompanying materials into a package that has the form of a bag 604. The robots 101, 102 can have six degrees of movement or more. The first robot 101 can be mounted on a guide to move longitudinally in the pharmacy area for accessing additional stages or areas of stages beyond the reach of an arm of the first robot 101. The first robot 101 can have at least seven axes of movement. The first robot 101 can be a unit-of-use robot that retrieves a unit-of-use product 602, which is still in its original packaging, from an environmentally controlled drug storage (for example, a refrigerated room) 105, applies a new label to the unit-of-use product 602 at a labeler 106, and delivers the unit-of-use product 602 into the bag 604 that is being held open at a packager 110. The second robot 102 operates to retrieve paper work 612, a temperature sensor 610, and a coolant pack 608.

The robots 101, 102 each include end of arm tooling adapted to engage the unit-of-use product 602 through, for example, grasping, suction, etc. The end of arm tooling includes various bag and box gripping solutions for various packaging such as bags, blister packs, boxes, bottles, ampules, syringes, medication containers and the like. The robots 101, 102 can each include one or more end of arm tools. By way of example, a first end of arm tool is adapted to engage a unit-of-use product 602, a second end of arm tool is adapted to engage an envelope from an envelope producer 111, a third end of arm tool is adapted to engage paperwork 612 from a paperwork producer 109, a fourth end of arm tool is adapted to engage a temperature sensor 610 from a temperature sensor feeder 108. The robots 101, 102 can be collaborative in that they can be adapted to physically interact with humans in a shared workspace and can be 6 or 7-axis robots.

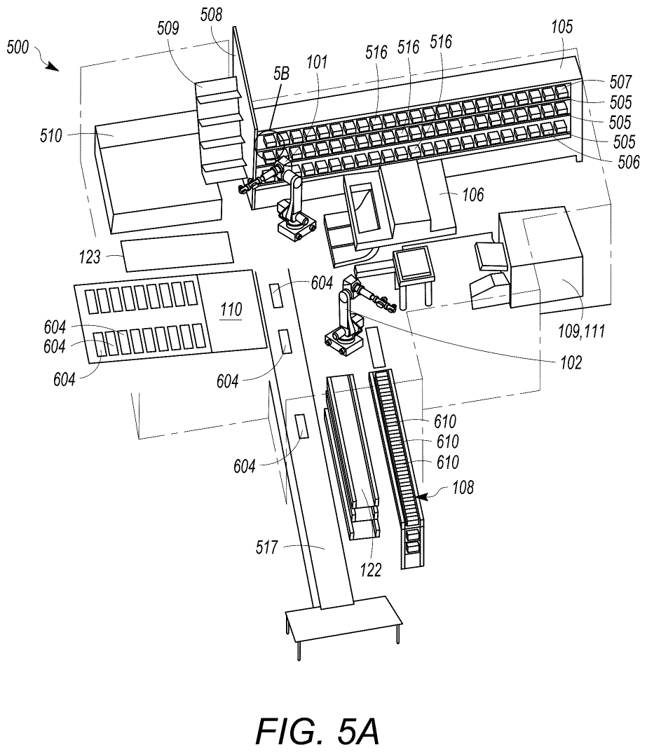

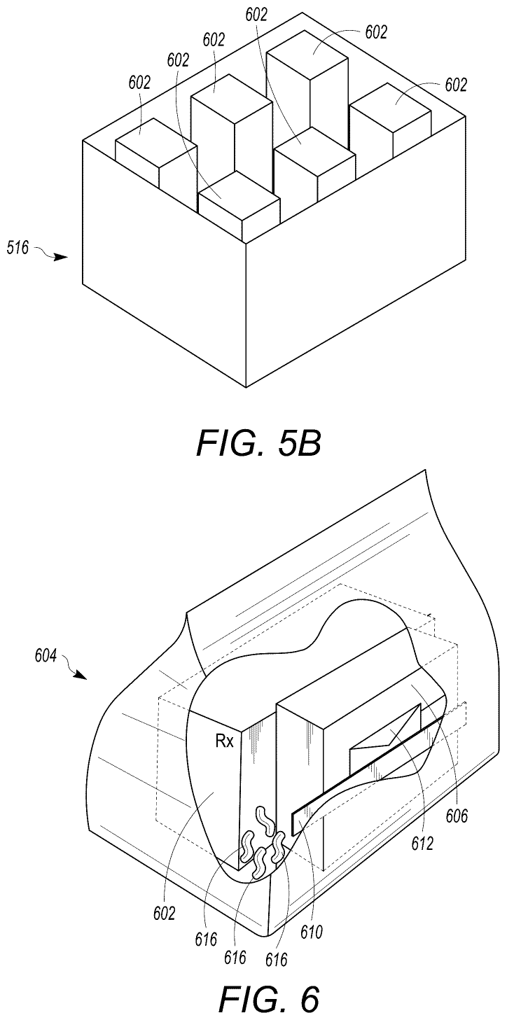

The environmentally controlled storage 105 holds at least one type of unit-of-use drug 602 at its required environment. The storage 105 can be a refrigerated cooler that has an internal temperature within the temperature range for each medication type stored therein, e.g., between 36-40° F. (2-8° C.). A single temperature excursion up to 77° F. (25° C.) may be allowed in some instances. After a temperature excursion, the drug should be returned immediately to the recommended storage condition 36-40° F. (2-8° C.) and used as soon as feasible. In some instances, the drug may need to be disposed when an excursion to a certain temperature or number of excursions occurs. In the embodiment of FIGS. 5A and 5B, the unit-of-use drugs 602 are contained in their original packaging that is itself within in original shipping containers (e.g., boxes 516) that have been opened.

When multiple medication types having different storage temperature ranges are stored, the storage 105 can have different zones that maintain different temperatures. The storage 105 can include a first, long-term storage area and a second, reach-in storage area. The first storage area can be an enclosed, insulated room that is accessible through a latchable door in the room defined by insulated walls, floor and ceiling. A refrigeration unit keeps the room at a set temperature using a temperature controller. The second storage area can have an open side through which the drug packaging robot 101 can retrieve a unit-of-use product 602 of a prescribed medication or other drug, e.g., a box containing a nasal mist drug. The second storage area can have movable conveyors therein to move a certain unit-of-use product 602 to the open side of the storage 105 so that it is graspable by the drug packaging robot 101 directly from the boxes 516. The second storage area can include a second refrigeration unit to keep the second storage area at a set temperature using a temperature controller. The second storage area can include a curtain at its open side to reduce the temperature loss through the open side. The first robot 101 can reach through the curtain to pick the medication or drug from the second storage area. The robot's arm can move the curtain or cantilevered fingers of a curtain while the arms moves into the storage area in the cooler.

The storage 105 can include multiple platforms to hold the unit-of-use products 602 above the floor. Prior to the boxes 516 containing the unit-of-use products 602 being placed on the platforms, the boxes 516 can be cut open to reveal the unit-of-use products 602 in their original packaging. This allows the first robot 101 to reach into the box 516 and directly engage one of the unit-of-use products 602 and remove it from the box 516 in a process sometimes referred to as “picking.” In some embodiments, the boxes 516 can be cut open manually by a person. In other embodiments, a robot can be programmed to automatically cut the boxes 516. In some embodiments, the boxes 516 are cut to only remove their tops, e.g., FIG. 5B. In some other embodiments, both the tops and some portions of one or more of the sides are removed.

By way of example, the platforms can be slides that have an entry end at which unit-of-use products 602 are inserted and an exit end at which the first robot 101 can remove a unit-of-use product 602. In an example embodiment, the entry end is higher than the exit end. Thus, the boxes 516 containing the unit-of-use products 602 on a platform can slide toward the exit end under the force of gravity overcoming the friction between the platforms and the boxes or unit-of-use products. The platforms can also include rollers that reduce the friction on the slide so that the boxes 516 containing the unit-of-use products 602 more easily fall toward the exit end. In some embodiments, a spring-loaded pusher can be located be at the rear of the unit-of-use products 602 on any individual platform to urge the boxes 516 containing the unit-of-use products 602 to the exit end. The side of the storage 105 where the exit end is positioned can be open to allow the first robot 101 access to the unit-of-use products 602 at the exit end. The platforms can include conveyors that are at least partially positioned in the long-term storage area so that the conveyor can move a box 516 containing a selected unit-of-use product 602 to a location that is reachable by the first robot 101 so that the first robot 101 can reach into the box 516 and retrieve a unit-of-use product 602 directly from the box 516. The conveyors include a moveable surface that is moved by a motor under direction of a controller to position a box containing a unit-of-use product 602. The storage 105 can vent chilled air at the exit end to form a temperature curtain at the exit end. The other sides of the storage 105 can be enclosed to maximize the efficiency of the refrigeration unit. The side of the storage 105 with the entry end can include moveable doors to selectively enclose that side of the storage 105 while allowing access to load the storage 105 with unit-of-use products 602 when open and reduce thermal conductivity and efficiency losses when closed. The platforms can include sensors that sense the level of unit-of-use product 602 on each slide and report inventory in the cooler to a central processor. A waste area 510 is located at the exit end of the storage 105 for receiving empty boxes 516 that have been emptied of their contents by the first robot 503. The waste area 510 can be a recycling bin for recycling the empty packaging.

The storage 105 is environmentally controlled, e.g., temperature and/or humidity controlled. The storage 105 can be a cooler. The storage 105 includes a plurality of tiers 505 (three being shown in the exemplary embodiment) that are separated from one another by longitudinally extending walls and that store the unit-of-use products 602 prior to them being selected by the robot 503. In the exemplary embodiment, the tiers 505 all hold the same unit-of-use products 602, but in other embodiments, different unit-of-use products 602 can be contained on the different tiers 505. In some embodiments, the fewer or more than three tiers 505 may be provided, and some of those tiers 505 may contain similar unit-of-use products 602.

The storage 105 can be at least partially open at a side 506 facing the first robot 101 so that the first robot 101 can reach into the storage 105 through the open side. In an exemplary embodiment, at least one blower can be positioned at the side 506 to blow a curtain of air along the side 506. The curtain of air separates the climate-controlled air within the storage 105 from the ambient air outside of the storage 105, thereby allowing the storage 105 to maintain a more consistent temperature, even adjacent the open side 506. The tiers 505 can be loaded from the rear of the storage or from a first end 507 of the storage 105. In the exemplary embodiment, the tiers 505 are sloped from the first end 507 towards a second end 508 located near the first robot 101 such that the previously-opened boxes 516 containing the unit-of-use products 602 are guided along the tiers 505 towards the first robot 101 with the assistance of gravity. In other words, the first robot 101 can engage any of the unit-of-use products 602 directly from its shipping box 516 at the second end 508 and, when one of the boxes containing the unit-of-use products 602 is empty and removed from the storage 105, the other products 602 in that tier 505 will automatically fall under the influence of gravity towards the second end 508, thereby putting a next sequential one of the boxes containing the unit-of-use products 602 in a ready-to-pick position adjacent the first robot 101. In some embodiments, the tiers 505 may include conveyors for delivering the boxes 516 containing the unit-of-use products 602 to the area of the first robot 101.

In the exemplary embodiment, a second end extension 509 is positioned at the second end 508 of the storage 105 and can engage empty boxes 516 and deliver those boxes 516 (or any other waste packaging) to a waste area 510, which is positioned adjacent the second end 508 of the storage 105.

In some embodiments, the cooler storage rack 122 can be positioned within the storage 105 or within an additional environmentally controlled storage space and in a location that can be accessed by either the first robot 101 or the second robot 102.

In some embodiments, one or both of the first and second robots 102 may be located within reach of the waste area 510 so that the robots 101, 102 can deposit waste (for example, faulty cooling packs, packaging, or boxes) into the waste area 510.

A labeler 106 is positioned in the arc of movement of the first robot 101. The labeler 106 can print a label with prescription data and adhere the label to a selected unit-of-use product 602 with the assistance of the first robot 101.

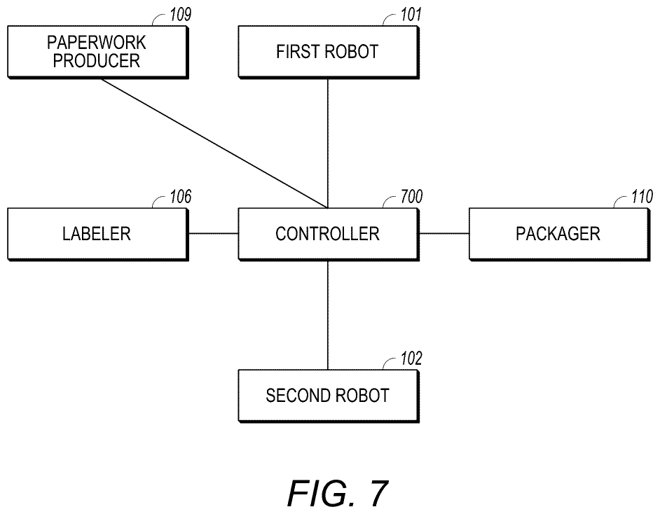

A temperature sensor feeder 108 is positioned in the arc of movement of the second robot 102. The temperature sensor feeder 108 provides a sensor 610 that can be packaged in the bag 604 with the unit-of-use product 602. In some embodiments, the temperature sensor 610 may not be included in the shipping package 600. The controller 700 (shown in FIG. 7) will track those unit-of-use products 602 that require the temperature sensor 610 and, if required, will instruct the second robot 102 to retrieve the temperature sensor 610 from the temperatures sensor feeder 108 and place it in the bag 604 but not for other prescriptions when not required. In one embodiment, the temperature sensor feeder 108 can include a plurality of different types of temperatures sensors 610, which are adapted for use with different types of medications that have different temperature range and time requirements.

A paperwork producer 109 is positioned in the arc of movement of the second robot 102. The paperwork producer 109 provides paperwork to be packages with the unit-of-use product 610. The paperwork may include, for example, drug information and/or dosage instructions.

An envelope feeder 111 is positioned in the arc of movement of the second robot 102. The envelope feeder 111 provides an envelope 612 in which the paperwork is stored. The envelope 612 can be packaged with the unit-of-use product 602 in the package 604. The envelope feeder 111 and the paperwork producer 109 can be part of a single paperwork system to produce the paperwork. For example, in one embodiment, the envelope feeder 111 can feed an envelope to the paperwork producer 109, which folds the paperwork and places it in an empty envelope 612 and feeds the assembled envelope 612 containing the paperwork to the second robot 102.

A packager 110 is positioned in the arc of movement of the first and second robots 101, 102. The packager 110 can be a top load bagger that retrieves and opens a package 604 and holds the package 604 open while the first and second robots 101, 102 can load the labeled unit-of-use product 602 and other components (for example, a temperature sensor 610, and/or an envelope 612 containing paperwork) into the package 604. The package 604 can have the form of a bag, and construction of the package 604 is discussed in further detail below. In some embodiments that are discussed in further detail below, the packager 110 may also make, or build, the package 604.

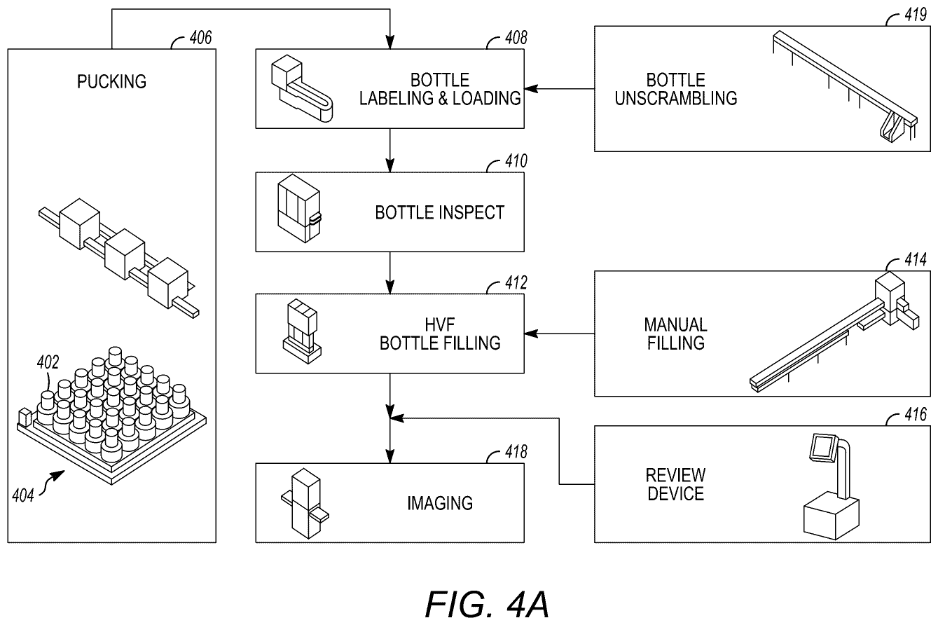

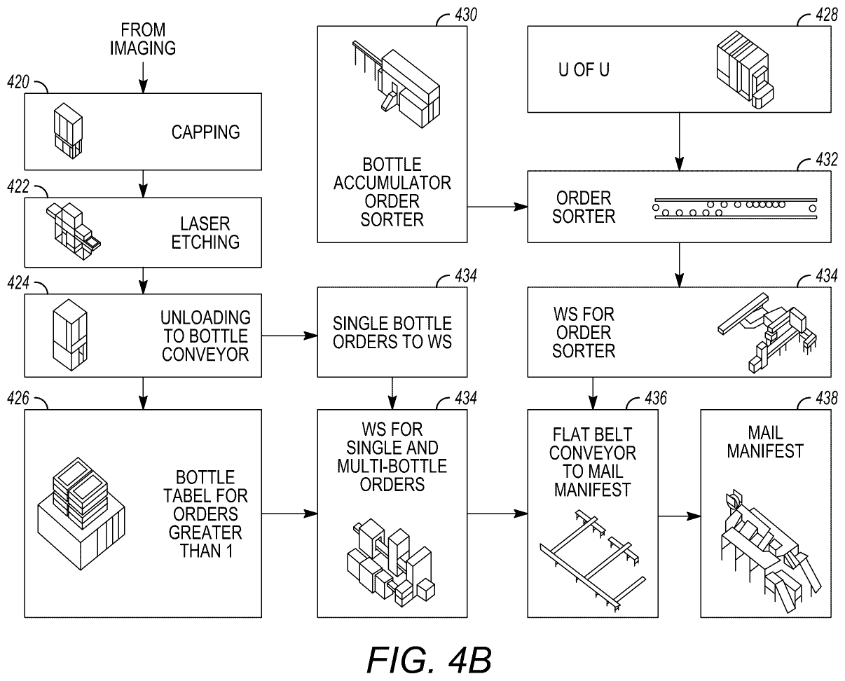

After the package 604 is filled with its contents by the first and second robots 101, 102 at the packager 110, the packager 110 seals the package 604 closed and places the package 604 onto a conveyor 517, e.g., a flat belt conveyor. The conveyor 517 transports the filled package 517 to a packaging device 434 to the shipping manifest 438 (schematically illustrated in FIG. 4B). In an exemplary embodiment, the packager 110 seals the package with a heat seal which applies heat to two flaps on the package 604 to seal those flaps together and trap the contents within the package 604. In an exemplary embodiment, the packager 110 can select from different sizes of packages 604 depending on the number of unit-of-use products 602 and the size of the coolant 606 to be contained therein.

In some embodiments, the package 604 can be sealed by a thermally activated seal or a mechanical seal formed between two opposed edges of the package 604. In an example embodiment, the package 604 includes an adhesive on at least one side that adheres the two sides of the opening together to close the opening in the sealed package 604. The package 604 includes an insulation so that it can act as a moisture barrier between the environment outside the package 604 and inside the package 604. The package 604 can be a flexible package, in an example embodiment. The flexible package 604 can have a least one non-rigid or stiff side. In an example embodiment. All sides of the package 604 are flexible. The package 604 can have an internal moisture barrier between the drug and the environmental control.

A coolant supply 122 is positioned in the arc of movement of the second robot 102 and includes coolants 606 (for example, ice packs or gel packs or any suitable means for controlling temperature in the shipping package 600) to the second robot 102 for inclusion in the package 604. The second robot 102 can select the size or amount of coolant 606 to be placed with the order package in package 604 at the packager 110. The coolant supply 122 may include different types and/or quantities of coolants 606 that have different cooling capabilities. For example, the coolants 606 may be able to maintain the contents of the shipping package 600 at different temperatures and for different periods of time based on, for example, the type of unit-of-use product 602 and its intended destination and the projected weather that the package 604 is to experience while at and on the way to the intended destination. In some embodiments, the coolant 606 may be sweat-resistant to resist condensation that could damage the packaging of the unit-of-use drug 602.

A filler supply 123 is positioned in the arc of movement of the first robot 101 for supplying a filling material 616 to the packager 110 for inclusion in the package 604. The controller directs the first robot 101 to select the appropriate size and/or amount of filling material 616 and coolant 606 to be placed with the package 604. The filling material 616 can be, for example, air bags, bubble wrap, packing peanuts, or the like.

A communication network 130 can provide instructions to the robots 101, 102 to fill a prescription order requiring a climate-controlled drug, which can be stored in the environmentally controlled drug storage 105. A user device 131 can submit a prescription order through the network 130. The user device 131 can include encryption to safeguard the prescription data. A pharmacy management device 133 is in communication through the network 130 and can receive the prescription. The pharmacy management device 133 can adjudicate the prescription claim and, if an automated pharmacy flag exists in the records associated with the patient, stored in electronic memory, the device 133 can instruct an order processing device 134, e.g., through the network 130, to fill the prescription at an automated pharmacy, e.g., pharmacy 100. The order processing device 134 can track the types of drugs that are stored in the climate-controlled storage 105 and if in the storage 105 and part of the prescription, instruct the robots 101, 102 to fill and package the climate-controlled drug as part of the prescription order.

A code reader may further be provided in the arc of movement of the first robot 101. The code reader can include a camera or a laser reader and processor to recognize an image from the camera or to convert reflected light from a unit-of-use product 602 into a digital code to be stored in memory and compared to a record in memory. The code reader can be a bar code reader, an alphanumeric reader, a quick-read (QR) reader, a radio frequency identification (RFID) reader, or the like. The first robot 101 can move an engaged unit-of-use product 602 to the code reader to confirm the correct unit-of-use product 602 is selected and compare the selected unit-of-use product 602 to the prescription order prior to placing the unit-of-use product 602 in the package 604.

The first and second robots 101, 102 operate to feed components, including the unit-of-use product 602, a temperature sensor 610, the coolant 606, and paperwork 612 (e.g., dosage information) associated with the prescription to the packager 110 and into the package 604. The packager 110 seals these drug order components within the package 604. The packager 110 can also operate to reduce or expel air from the package 604 prior to sealing the package 604.

In some embodiments, the system 100 can all be contained within a temperature-controlled environment. The automation of the entire system 100 means that the system 100 can be maintained at the required temperature for the unit-of-use product 602, which may not be a temperature at which a person can work for any significant period of time.

The system 100 selects the amount and/or type of coolant 614 (for example, one or more gel or ice packs) to ensure the unit-of-use product 602 is maintained within the temperature range until it reaches its final destination. In an example embodiment, the coolant 614 is a “sweat-proof” gel pack that is adapted to trap any condensation that might be produced so that the condensation does not damage the unit-of-use product 602 or the other contents within the package 604. This can be done using the systems and methods described in U.S. patent application Ser. No. 14/630,373, titled Methods and Systems for Prescription Drug Shipping Selection, filed 24 Feb. 2015, which is incorporated by reference herein. Additional methods and systems for using ice or other coolant are described in U.S. patent application Ser. No. 11/818,330, titled Containers for Transferring Products and Methods for Their Transfer, filed 14 Jun. 2007, is hereby incorporated by reference.

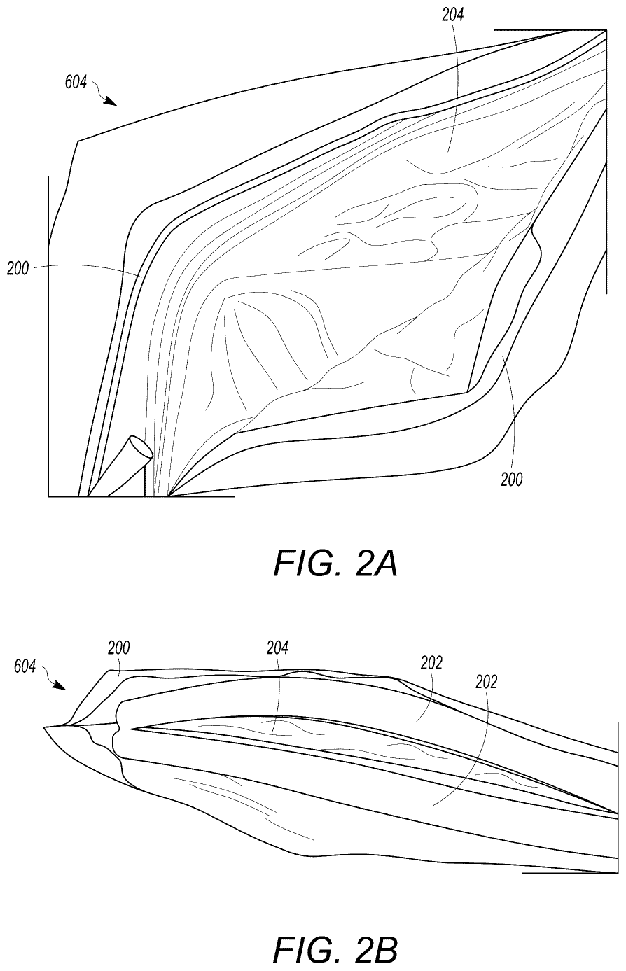

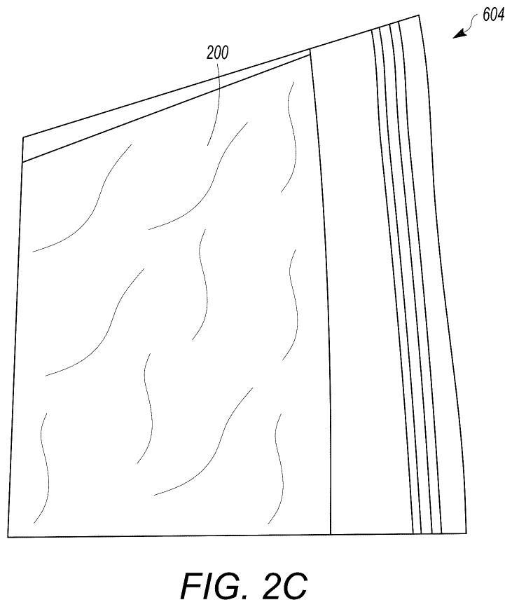

FIGS. 2A, 2B, and 2C are images of an example embodiment of a package 604. The package 604 includes three layers: an outer layer 200, an insulating layer 202, and an inner layer 204. The outer layer 200 is made of two sheets of a metal foil, e.g., aluminum foil. The sheets of foil are bonded together along portions of their respective peripheries to give the outer layer 200 a pouch-shape with an open interior. The middle, insulating layer 202 is made of two pieces of foam that are inserted into the interior of the outer layer. Any suitable type of thermally insulating foam may be employed for the insulating layer 202. In some embodiments, the foam pieces of the insulating layer 202 are not bonded to the foil sheets of the outer layer 200. In other embodiments, the foam pieces of the insulating layer 202 are adhesively attached with the outer layer 200. The inner layer 204 of the exemplary embodiment has the form of a plastic pouch that is inserted into the space between the foam pieces of the insulating layer 202.

In some embodiments, the system may be provided with packages 604 that are differently configured. For example, some packages 604 may be larger than others to accommodate additional unit-of-use products 602 and/or additional coolant 606. Some packages 604 may contain more of the insulating layer 202 (for example, more or thicker pieces of foam or a different type of foam) than other packages 604 to keep the unit-of-use product 602 contained in the package 604 within the proper temperature range for longer and/or in more extreme environments (for example, extreme hot or cold temperatures).

In some embodiments, the packager 110 or a separate bagging machine builds the package 604 prior to the robots 101, 102 loading the unit-of-use product 602 and the other contents into the package 604. In one such embodiment, the packager 110 has access to outer layers 200 of different sizes; insulating layers 202 of different sizes, thicknesses, and/or materials; and inner layers 204 of different sizes. The controller uses a number of inputs to determine which outer layer 200, insulating layer 202, and inner layer 204 to select when building the package 604. The inputs may include, for example, the size/number of unit-of-use products 602 to include in the package 604, the estimated delivery time, the projected temperature at the destination at the estimated time of delivery, and the projected temperatures during the delivery path. For example, the controller may direct the packager 110 to build a package 604 with maximum insulating layer 202 thickness if the package 604 is being delivered to Phoenix, Arizona during the summer.

An insulated bag provides resistance to heat flow and assists in keeping the contents therein within its thermal range or temperature requirements. This can reduce waste and increase the safety of drugs delivered via common carriers, e.g., USPS, FedEx, UPS, Lyft, Uber or the like. An insulated bag can assist in controlling its internal volume by reducing heat flow, e.g., at least one or more of conduction, convection, and radiation. Conduction is heat moves through materials, such as through inner layer, outer layer and the insulation layer of the bag. Convection can occur within the bag as air flows around in the interior of the bag. Radiant heat travels in a straight line through the solid layers of the bag. In an example, the insulation layer traps air and material between the inner and outer layers, this can reduce all three types of heat transfer from outside the bag to the interior of the bag. At least one of the sides of the inner layer and the outer layer can be reflective, e.g., in the red or infrared spectrum to reduce heat transfer. To be most efficient the reflective layer should face an air space, e.g., on outer surface of outer layer, inner surface of inner layer, or on the insulation layer. To maintain the interior volume of the bag with the desired temperature range, heat intrusion can be adsorbed by a gel pack or ice pack in the interior of bag.

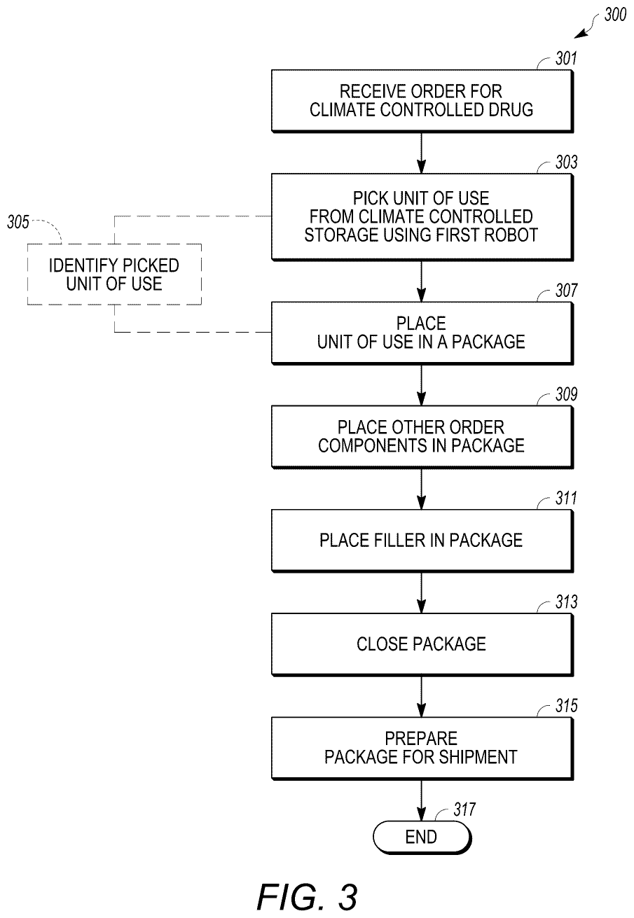

FIG. 3 is an example process flow illustrating a method 300 for filling a prescription order including an environmentally controlled drug in an automated pharmacy, according to an example embodiment. In various example embodiments, the system 100 can execute the method 300 or parts of method 300.

At block 301, an order for a drug that is climate controlled is received, e.g., at a controller or control circuitry at an order fulfillment center. The drug is a unit-of-use product 602. At block 303, the unit-of-use product 602 is picked from the climate-controlled storage 105 by a first robot, e.g., the first robot 101. At block 305, in some embodiments, the picked unit-of-use product 602 is identified, e.g., by the first robot moving the unit-of-use product 602 to a code reader 202 (such as a laser reader). At block 307, the picked unit-of-use product 602 is placed in a package 604. At block 309, other components (for example, an envelope 612 containing paperwork, a temperature sensor 610, etc.) are placed in the package 604. At block 311, the filler material 616 is added to the package 604. The preceding steps that require movement of material can be performed by the first robot 101 and/or the second robot 102.

At block 313, the package 604, containing the unit-of-use product 602 and other components, is closed by the packager 110. At block 313, a shipping container (such as a cooler 120) is selected for the closed package.

At block 315, the package 604 is moved to a shipping finisher 125 and the package 602 is prepared for shipping, e.g., tape or shrink wrap, then label for the mail carrier or shipping company. In an example embodiment, at the shipping finisher 125, the package 604 can be thermally sealed or otherwise closed to trap the unit-of-use product 602 and other contents therein. This will reduce or prevent convention of air between the interior of the bag and the environment. At block 317, the process ends. Ending results in a single prescription being packaged and shipped to a customer. The process steps can be performed in the order described above, another order, or performed simultaneously for different prescriptions. The end can also signal to the controller 700 that the system 100 is awaiting a further prescription to fill.

FIG. 4A is a diagram of an example implementation of a system for an automated pharmacy, according to an example embodiment.

In an automated or high-volume pharmacy (HVP) system generally designated 400 and shown in FIGS. 4A and 4B, an order is generated that can include one or more components including, for example, prescription drugs and/or supplements (generally product), for fulfillment. Generally, the order components include containers, unit-of-use products, or other packaging having a quantity of a prescription drugs, supplements, or the like therein. The unit-of-use products can be environmentally controlled, e.g., stored in cooler. The components of a given order can be separated in time and space within the system 400 and are therefore routed to various holding areas and processing areas within the system 400 so that all of the order components of an order can be joined together, or “married”, for packing and further processing. The system 400 can include the system 100 (shown in FIG. 1) to handle environmentally controlled components of an order. The system 400 can join the enclosed shipment package 600 (shown in FIG. 6A) with other order components in an example. In another example, the components of an order can be processed separately but arrive at the outgoing shipment from the pharmacy at the same time or ready for delivery to the carrier at the same time.

Note that the pharmacy described herein is an automated pharmacy, e.g., a high-volume pharmacy, in contrast with a retail pharmacy or a specialty pharmacy, for example. While the system 100 is described as being deployed in a high-volume fulfillment center (e.g., a mail order pharmacy, a direct delivery pharmacy, and the like), the system 100 and/or components thereof can be deployed in a retail pharmacy or a specialty pharmacy, for example.

### Pucking

The system 400 transports containers 402 (e.g., between devices described in the system 400) by use of pallets 404. A pallet sizing and pucking device 406 configures pucks in a pallet. The term “puck” is meant to describe a receptacle sized shaped and configured to receive a specific container 402. There can be many different sizes of containers and a corresponding puck can be needed to hold the container in the pallet receptacle. A puck is placed in a cavity in a pallet 404 by the pallet sizing and pucking device 406. Containers 402 are supported by the pucks during carriage in the pallet 404. Different pucks can have differently sized and shaped receptacles to accommodate containers 402 of differing sizes and shapes. The pallet 104 defines a transport structure for a number of pucks and the containers 102. The pallet 104 can include a number of cavities that each receive a puck and/or a container 102.

In some example embodiments, the pallet includes receptacles sized to receive the containers and need not include pucks to size the pallet receptacles to receive the containers 402. The use of a single container can allow for pallets to have receptacles sized for the single size container 402.

### Bottle Loading and Labeling

The automated pharmacy system 400 also includes a loading device 408 for loading containers 402 into the pucks on a pallet 404 by for example, a robotic arm, pick and place mechanism, or other suitable device. The loading device 408 can also print a label (not shown) appropriate for a container 402, which is to be loaded onto the pallet 404, and apply the label to the container 402. The pallet 404 can be located on a conveyor assembly during these operations, e.g., at the automated pharmacy system 400.

### Unscramble Device

A container unscrambling device 419 of the pharmacy system 400 is used to take a container of empty containers 402 either from a box, a bin, or other container, and orient the containers 402 in the correct position and load the containers 402 onto the infeed conveyors for further processing within the system 400.

### Bottle Inspect

The pharmacy system 400 also includes a bottle inspect device 410 configured to verify, among other functions, that containers 402 in pallet 404 are correctly labeled and in the correct position on the pallet 404. The inspect device 410 suitably scans a label (barcode, text, or other suitable image) on container 402 on the pallet 404. Labels of the containers 402 can be scanned or imaged in full or in part by the inspect device 410. Such imaging can occur after container 402 has been lifted out of its puck by a robotic arm, picker, or the like, or can be otherwise scanned or imaged while retained in the puck. In some embodiments, images and/or video captured by the inspect device 410 can be stored in a storage device as order data.

### Auto HVF Filling

An automated high-volume fill (HVF) bottle filling device 412 dispenses ordered medications or similar things into containers 402 in accordance with associated orders. HVF bottle filling device 412 includes high volume fillers that fill a number of drug types at a rapid rate. High volume fillers store a significant quantity of medication units, e.g., pills, tablets, gel caps, capsules, and the like, and count and dispense an ordered quantity into a container assigned to an order. The device 412 can be automated. Orders dispensed by the HVF bottle filling devices 412 can be packaged individually or in a container for shipping or can be shipped in combination with other orders dispensed by other devices in the pharmacy system 400. The other orders can be the environmentally controlled orders as described herein. Within the HVF bottle filling device 412 are secondary staging devices (not shown) that allow the orders to be pre-staged per order prior to the container 402 actually arriving at the dispense position or lane/area.

### Manual Filling

A manual fulfillment station 414 is configured for manual fulfillment of orders. For example, manual fulfillment station 414 is configured to enable fulfillment of drugs in the container 402 by a pharmacist or pharmacy technician. In some embodiments, manual fulfillment station 414 provides the filled container 402 to another device in the pharmacy system 400 to be joined with other containers 402 in an order for a patient or member.

Orders dispensed by manual fulfillment station 414 are packaged individually or collectively for shipping. The order component from the manual fulfillment station 414 can be shipped in combination with other products dispensed by other devices in the pharmacy system 400, e.g., an environmentally controlled order component from system 100.

In general, manual fulfillment includes operations at least partially performed by a pharmacist and/or pharmacy technician. For example, manual order filler at the station 414 retrieves a supply of a prescription drug, makes an observation, counts out a prescribed quantity of drugs and places them into container 402, or the like with at least part of this operation being manual. Some portions of the manual fulfillment process can be automated by use of a machine. For example, counting of capsules, tablets, or pills can be at least partially automated (e.g., through use of a pill counter). The manual fulfillment station 414 also includes multiple devices (not shown) that capture an image of the medications and/or weighs the medications to aid the pharmacist or technician in counting and verifying that the correct count has been reached.

### Review Device

A review device 416 processes containers 402 to be reviewed by a pharmacist for proper pill count, exception handling, verification, and the like. Fulfilled prescriptions reviewed and/or verified by a pharmacist, as can be required by state or local law, either manually or using a review device 416. A pharmacist dispenses certain drugs in compliance with local and/or other laws and operates the review device 416 to visually inspect a container 402 that has been filled with a prescription drug. The pharmacist reviews, verifies, and/or evaluates drug quantity, drug strength, and/or drug interaction concerns, or otherwise performs pharmacist services. The pharmacist also handles containers 402 that have been flagged as an exception, such as containers 402 with unreadable labels, containers 102 for which the associated order has been cancelled, containers 402 with defects, and the like.

### Imaging

The pharmacy system 400 also includes an imaging station 418 that includes an imager (for example, a camera), which can image containers 402 after they have been filled with product, e.g., a medication. The imaging station 418 measures the fill height of the product in the container 402 based on the obtained image to determine if the container 402 is filled to the correct height given the type of product and the number of pills in the prescription. Images of the pills in the container 402 can also be obtained to detect the size of the pills themselves and markings thereon. The images can be transmitted to the order processing device 400, and/or stored in the storage device 402 as part of the order data 404.

### Capping

A capping station 420 is used to cap or seal container 402. In some embodiments, the capping station 420 includes a capping device to secure container 402 with a type of cap in accordance with a preference (e.g., a preference regarding child resistance that can be defined by for example, a patient, plan sponsor, or a prescriber). The cap device 420 applies the caps to a predetermined torque standard that allows for easy removal. Cap-maps are used to validate that the proper torque has been reached and, if not, a secondary inspection by a pharmacist is required.

### Laser Etching

The pharmacy system 400 also includes a laser etching station 422 that etches a message and/or image into the cap. In an example embodiment, the etching station 422 can be physically integrated with the capping station 420. In addition to laser etching, etching station 422 can include other forms of marking the top of the container 402. For example, station 422 can mark the tops of the containers 402 with ink or labels that have been pre-marked with ink or otherwise.

### Unloading

The pharmacy system 400 also includes an unloading station 424. Generally, there are two types of unloading. The first is smart unloading, which matches the container 402 to an order or literature in sequential order that pertains to the same order for multiple containers. The second unloading type is non-sequential unloading that allows the container 402 and literature to be married up at a later time.

### Bottle Table

Bottle tables 426 are used for vertical storage of containers 402 that are sequentially parts of the same order. The bottle table 426 will store up to four containers 402 and is used to marry up parts of an order that can be processed at different locations, times, or areas of the pharmacy system 400. The purpose then is to bring all the containers 402 together and release the containers 402 together as an order in a sequential fashion for further processing or preparing the order for shipping.

### Unit-of-Use

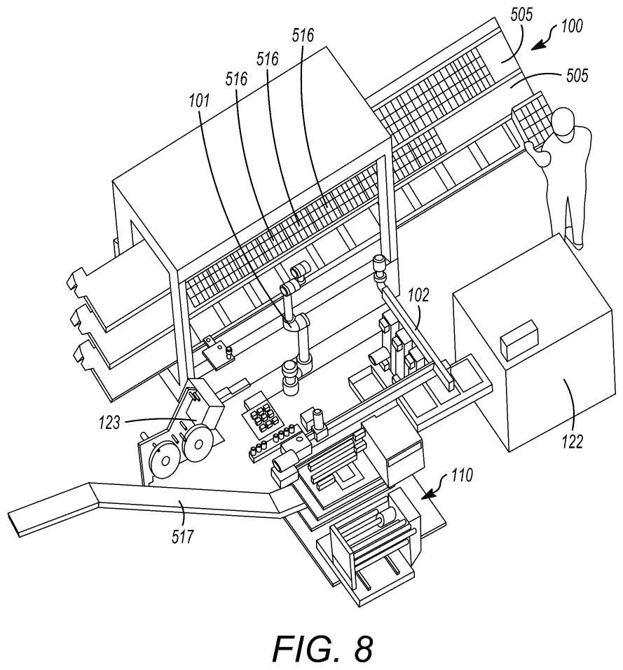

The system 400 includes a unit-of-use station 428 that temporarily stores, monitors, labels and/or dispenses unit-of-use products. In general, unit-of-use products are products that are delivered to a patient or member without being repackaged (e.g., after receipt from a pharmaceutical manufacturer or distributor) at the pharmacy. These products can include pills in a bottle or bottle-like container; pills in a blister pack; inhalers; gels; and the like. Products dispensed by the unit-of-use station 428 can be packaged individually or collectively for shipping or can be shipped in combination with other orders dispensed by other devices in the pharmacy system 400. The unit-of-use station 428 can include the system 100 shown in FIG. 1 or system 500 shown in embodiments in FIGS. 5A and 8 for handling environmentally controlled drug order components, e.g., unit-of-use products 602 that need to be cooled until use. The unit-of-use station 428 can also include a separate system for processing unit-of-use products that are not environmentally sensitive.

The unit-of-use station 428 is used to take the manufacturer's bottles, boxes, or containers, and load them into a system that can sort, bag, or combine them with other equipment in the pharmacy, to complete the order. The end result of the unit-of-use station 428 is to have the bottle or container/box bagged and processed for mail delivery. The unit-of-use station 428 can at least partially operate in an environmentally controlled environment, e.g. a temperature-controlled setting that stores and process drugs to maintain the drug in the temperature range during processing and during delivery.

### Accumulator

The accumulation station 430 accumulates various containers 402 in an order. The accumulation station 430 can accumulate containers 402 from various devices or areas of the pharmacy. For example, the accumulation station 430 can accumulate container and/or products from the unit-of-use station 428, the HVF filling station 412, the manual fulfilling station 414, and the review device 416, at the pharmacy system 400. The accumulation station 430 is used to group the containers and/or products prior to shipment to the member or otherwise. The accumulation station 430 shown in FIG. 4B is considered to be a horizontal accumulator, whereas the bottle table 426 can be considered to be a vertical bottle accumulator. The accumulation station 430 can receive an order component from the system 100, which is enclosed with coolant sufficient to allow delivery of the order via mail, other common carrier, courier, drone delivery, or otherwise within the temperature range.

### Order Sorter

The order sorter 432 receives containers 402 from the accumulation station 430 and combines the order components, e.g., unit-of-use products, bottles, vials, and the like into an order that can be packaged and shipped in the mail system.

### Packaging

The pharmacy system 400 also includes a number of wrap seal or packaging devices 434 that package the various components of an order together. The packing device 434 packages an order in preparation for shipping the order. The packing device 434 boxes, bags, sealed coolers or otherwise packages the fulfilled order for delivery. The packing device 434 also places inserts into the packaging. Bulk orders can be shipped in a box, while other orders can be shipped in a bag, which can be a wrap seal bag. The packing device 434 can label the box or bag with the address and a recipient's name. The packing device 434 can sort the box or bag for mailing in an efficient manner (e.g., sort by delivery address). The packing device 434 can include ice or temperature sensitive elements for products which are to be kept within a temperature range during shipping in order to retain efficacy or otherwise.

As shown in FIG. 4B, the pharmacy system 400 includes multiple packaging devices 434 for packaging different types of orders at different stages of order processing. These packaging devices 434 can be different parts of the same device or can be different devices. Further, there can be more than one packaging device 434 at each depicted location of a packaging device 434. Generally, the packaging devices 434 packages containers 402 and other order materials from any combination of the automated dispensing device 412, the manual filling device 414, the review device 416, the imaging device 418, the cap device 420, the laser etching device 422, the unloading device 424, the container table device 426, the unit-of-use device 428, the accumulator device 430, and the order sorter device 432 at the pharmacy system 400, as well as the further packaging environmentally controlled drug orders from the system 100, 500 with other drug orders.

### Flat Belt Conveyor

After an order has been through the packaging device 434, a conveyor 436, e.g., a flat belt conveyor, transports the packaged order to from a packaging device 434 to the shipping manifest 438.

### Shipping Manifest

The shipping manifest station 438 receives orders from the packaging device 434 and then ships the package through a carrier, postal mail, through a mail order delivery service that ships via group and/or air (e.g., UPS®, FedEx®, or DHL®), through delivery service, through a locker box at a shipping site (e.g., Amazon Locker® or a PO Box), or otherwise.

The controller 700 for the first and second robots 101, 102 can track the order and the individual items for the order as well as the inventory and location of the items in the pharmacy 500. The controller 700 can send orders to the first and second robots 101, 102 to fill an order including the locations of the items to be picked by the robots 101, 102 and deposited into the package 604.

Turning now to FIG. 8, a second exemplary embodiment of the system 100 is generally shown with like numerals identifying corresponding parts with the above-referenced embodiment. The first robot 101 that picks items from the cooler is a rotational robot with an articulated arm that turns centrally on a vertical axis and can move linearly along a straight-line path in front of the cooler. The first robot 101 can be a 6-axis robot. The second robot 102 is a gantry mounted robot that moves along two linear gantries in a horizontal direction, respectively and has an end-of-arm tool that moves vertically to engage items and lift the items to move them to a workstation. The first and second robots can overlap in their travel and are coordinated such that they do not collide. The moveable components of the robots can include sensors that detect unexpected resistance to travel, e.g., contacting each other, which will cause the robots to stop movement. The robots can automatically reverse their respective movement to their prior action with an item or rest position. Then, the controller will decide which of the first and second robots will complete its task and then return to a position outside the travel of the other robot, which then completes its task. In an example, embodiment, sensor, e.g., imagers can be mounted above the robots in the systems 100, 500 to sense the position of the first and second robots as well as detect when an item or other object has entered the field of travel of the two or more robots. When an unexpected item (for example a person, box, dropped unit-of-use, paper, or the like), enters the field of travel of the robots, then the controller will stop the robots and signal an error condition.

The systsm 100, 500 show loading the cooler from a lateral side on the shelves 505, in another example, the shelves can be loaded from the rear. Side loading the shelves is shown as this allows the shelves to be tilted forward toward the robots, while being sloped from the load end to the finish end. The shelf can achieve these configurations while the load end is still at an accessible height, e.g., 6 feet (for the top shelf). The shelf also has a length such that the shelf can replenished with items, e.g., unit-of-use, from the same side as the robots with the replenishment area (see top right area of FIG. 8) being outside the area of travel of the robots (robots 101, 102 in FIG. 8).

The present disclosure describes some embodiments with regard to a unit-of-use product. A unit-of-use product is made for dispensing a medication to a patient without product packaging modification (or with minimal product packaging modification) except for labeling with patient information. Unit-of-use products can include a full course of medicine to be taken by a patient, for example, an entire prescription (e.g., a thirty-day supply, a sixty-day supply, or a ninety-day supply). The unit-of-use products contain known quantities of medication in packages that are closed and sealed by, for example, the pharmaceutical manufacturer. Dispensing unit-of-use products greatly limits the need for manual or automated filling of open prescription containers with medication units that are first counted and then sealed at the pharmacy.

Various embodiments described herein use two or more robots for order fulfillment. The robots are each responsible for their individual areas or volumes of action and interact with each other. The interaction can be overlapping areas (e.g., radius) of movement. For example, a first robot can retrieve a shipping container and a second robot can retrieve the object that is part of the order from an environmentally controlled storage. In an example embodiment, another robot can place coolant in the shipping container or place the lid on the container. In an example embodiment, the first robot or a third robot can thereafter move the shipping container storing the object to a shipping station. In an example embodiment, the shipping container storing the object to a shipping station is positioned on a conveyor that moves the filled shipping container to the shipping station.

Examples of a pharmaceutical order processing system, as described herein, can include a first pharmaceutical container storage area configured to support a first group of unit-of-use containers and a second group of unit-of-use containers. A container selector including a robot configured to pick one or more unit-of-use containers of the first group from the storage area and optionally pick from the second group. In an example, the robot can include a carriage supporting the arm, the arm being linearly moveable on carriage relative to the picking area of the storage area or the storage area. This allows the robot to pick from different locations relative to the storage area.

A consolidator robot can act as an order consolidator configured to receive the one or more pharmaceutical containers and the one or more unit-of-use containers.

A second pharmaceutical container repository configured to further supply a pharmaceutical containers, which can include non-environmentally controlled drugs. The consolidator robot is configured to receive one or more pharmaceutical containers and place the one or more pharmaceutical containers with the environmentally controlled unit-of-use containers. The second pharmaceutical container repository can be part of a high-volume filler configured to fill the pharmaceutical containers with the non-environmentally controlled drugs; The consolidator robot can move the one or more pharmaceutical containers past one or more identification sensors configured to scan the one or more pharmaceutical containers to verify the identity of the one or more pharmaceutical containers prior to consolidation with the unit-of-use. In an example embodiment, one of the first robot or the second robot includes a second end of arm tool that can engage the one or more pharmaceutical containers to consolidate them in a package with the picked unit-of-use, with the environmental control.

In an example embodiment, the carriage is movable in at least one of a generally horizontal direction or a generally vertical direction.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method can be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements but can also be an indirect relationship where one or more intervening elements are present (either spatially or functionally) between the first and second elements.

As used herein, the phrase at least one of A, B, and C should be construed to mean a logical (A OR B OR C), using a non-exclusive logical OR, and should not be construed to mean “at least one of A, at least one of B, and at least one of C.” The term subset does not necessarily require a proper subset. In other words, a first subset of a first set can be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information, but information transmitted from element A to element B is relevant to the illustration, the arrow can point from element A to element B. This unidirectional arrow does not imply that no other information is transmitted from element B to element A. Further, for information sent from element A to element B, element B can send requests for, or receipt acknowledgements of, the information to element A.

In this application, including the definitions below, the term “module” or the term “controller” can be replaced with the term “circuit.” The term “module” can refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module or other physical element in the pharmacy system can include one or more interface circuits. In some examples, the interface circuit(s) can implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4. For example, the interface circuits allow the robots to communicate with a controller, with each other or with other devices.

The module can communicate with other modules using the interface circuit(s). Although the module can be depicted in the present disclosure as logically communicating directly with other modules, in various implementations the module can actually communicate via a communications system. The communications system includes physical and/or virtual networking equipment such as hubs, switches, routers, and gateways. In some implementations, the communications system connects to or traverses a wide area network (WAN) such as the Internet. For example, the communications system can include multiple LANs connected to each other over the Internet or point-to-point leased lines using technologies including Multiprotocol Label Switching (MPLS) and virtual private networks (VPNs).

In various implementations, the functionality of the module can be distributed among multiple modules that are connected via the communications system. For example, multiple modules can implement the same functionality distributed by a load balancing system. In a further example, the functionality of the module can be split between a server (also known as remote, or cloud) module and a client (or, user) module.

Some or all hardware features of a module can be defined using a language for hardware description, such as IEEE Standard 1364-2005 (commonly called “Verilog”) and IEEE Standard 1076-2008 (commonly called “VHDL”). The hardware description language can be used to manufacture and/or program a hardware circuit. In some implementations, some or all features of a module can be defined by a language, such as IEEE 1666-2005 (commonly called “SystemC”), that encompasses both code, as described below, and hardware description.

The term code, as used above, can include software, firmware, and/or microcode, and can refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The robots described herein include memory circuits to store control instructions and order information. The term computer-readable medium, as used herein, does not encompass transitory electrical or electromagnetic signals propagating through a medium (such as on a carrier wave); the term computer-readable medium can therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application can be partially or fully implemented by a special purpose computer created by configuring a computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The processors can control the robots, or any other device described herein. The computer programs can also include or rely on stored data. The computer programs can encompass a basic input/output system (BIOS) that interacts with hardware of the special purpose computer, device drivers that interact with particular devices of the special purpose computer, one or more operating systems, user applications, background services, background applications, etc.

The computer programs can include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code can be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

The present systems and methods automate the dispensing of environmentally controlled drugs. A controller module, which may be located in the order processing device 134, can control robots, labelers, packagers, supply units and the like, to select, package and ship an environmentally controlled drug without the intervention of a human during these steps. The systems and methods can be part of a larger pharmacy, e.g., a specialty pharmacy or an automated pharmacy, but in an environmentally controlled part of such a larger pharmacy. The controller module can track an order from receipt from a medical care prescriber or a pharmacy benefits manager to delivery of a prepared package to the shipper. The prepared package includes the environmental controls for the drug.

The present system, as described herein, can include an environmentally controlled storage to hold drugs at a required environmental setting, a first robot to pick an ordered drug from the storage and package the drug, a second robot to receive the packaged ordered drug from the first robot and further package the packaged ordered drug for shipment with environmental controls to ensure that the ordered drug is maintained in the required environment during shipment from the automated pharmacy to the patient's location. The first robot can be configured to place the drug into a flexible container, e.g., a bag with at least two flexible sides. The bag can provide a moisture barrier between the drug and the environmental control, e.g., an ice pack, a gel pack or the like.

The environmentally controlled storage can be a temperature-controlled cooler with one or more temperature zones to store one or more temperature-controlled drugs within the required temperature range. The zones can have different temperature ranges. The cooler can store drugs at a lower temperature than room temperature and above freezing.

The first robot can be moveable in six dimensions and adapted to grip and move the ordered drug from the storage. The first robot can have a grasper hand or can rely on suction cups to engage the ordered drug, which can be a unit-of-use product. The first robot can move the ordered drug between multiple stations in the pharmacy. The stations can include bar code reader to check that the correct drug is selected from the storage. The ordered drug can then be moved to a labeler, which can apply a label to the ordered drug. The label can be printed at the labeler and adhered to the package. The label includes the type of drug, dosage, the patient name and address, pharmacy name, prescription number and other data. The first robot can move to other stations, such as a temperature sensing card dispenser, a sharps box feeder, a paperwork feeder, and envelope feeder and a packager. In an example, the ordered drug is moved to the labeler and then to the packager and placed in a package. The first robot can then retrieve the other order components from the other stations, i.e., the temperature sensing card dispenser, the sharps box feeder, the paperwork feeder, and the envelope feeder. The packager can then seal the ordered drug with the other order components in a package, e.g., a sealable bag.

The second robot has access to the packager and can retrieve a sealed package therefrom. The second robot can place the sealed package in an environmentally controlled determined structure that can ensure the ordered drug arrives at the patient location while maintaining the required environmental control. The second robot has access to a plurality of different shipment structures, i.e., differently sized coolers, different types or quantities of coolant. The second robot picks a correct shipment structure and places the sealed package therein. The second robot can then pick and place an ice pack or cooling gel in the shipment structure. Additional packaging can be added, if needed, to fill the shipment structure. A lid that is appropriate to the shipment structure is selected to enclose the sealed drug package and the coolant. The shipment structure is then secured for shipment, e.g., taped shut and labeled. The shipment package can be on a conveyor when secured and moved to a shipment site, which can or cannot be temperature controlled.

As described herein the first robot engages the drug to prepare a preliminary package including the drug and other order components. The second robot receives the preliminary package and packages that for shipment with the needed temperature control apparatus to ensure that the preliminary package is environmentally controlled during shipment. Shipment can be made through a common carrier once the order is packaged to provide the environmental control.

Example embodiments as described herein are directed to filling medication orders with environmentally controlled drugs. However, the systems and methods described herein may be adapted to complete orders for other environmentally controlled objects. An order is generated for an automated fulfillment system, e.g., a high-volume fulfillment system. A high-volume fulfillment system can include automatic object dispensing systems to carry out the dispensing of the objects automatically at a rapid rate. The objects can be ingestable or perishable. In the fulfillment system, an object can require environmental control while in storage and during shipping. The order can include more than one object for fulfillment. Each object in an order is an order component of the total order. An example of a controlled environment in the fulfillment system is one that maintains a required temperature range and/or humidity range. The controlled environment can be a lowered temperature. By way of example, a certain object requires a storage at a temperature less than room temperature but greater than freezing. The objects can be dispensed at various sections of the automated fulfillment system. Some orders can require manual handling of certain order components. Some order components can be filled automatically by filling machinery. The system can include at least two robots to retrieve, package, and prepare for shipping the order with an environmentally controlled object.

The controlled environment in which the object(s), e.g., perishables, drugs, pharmaceuticals, foodstuffs, and the like, are placed, e.g., by the robotics described herein, can include a shipping bag. The shipping back can be insulated, e.g., with multiple layers that resist heat transfer and the flow of heat. Insulated shipping bags can be made using a layer of insulation material, such as polystyrene foam, sandwiched between two layers of durable material, such as polymer, polyethylene or the like. This layered construction helps to keep the contents of the bag at a consistent temperature during transport, e.g., reduce conduction, convection, and/or radiation. The layers are usually sealed together using a heat-sealing process, which creates a strong and airtight seal. In some cases, the bags may also be equipped with additional features, such as handles or zippers, to make them easier to transport or to access the internal contents.

What is claimed is:

1. A temperature-controlled medication packaging system, comprising:
a storage including a temperature-controlled interior containing at least one medication that is disposed within a shipping box;
at least one shipping container in a packager;
at least one coolant that is sized to fit within the at least one shipping container; and
at least one robot adapted to retrieve the at least one medication directly from the shipping box within the storage and transport the at least one medication to the shipping container in the packager and to retrieve the at least one coolant and to transport the at least one coolant to the shipping container in the packager.

2. The temperature-controlled medication packaging system as set forth in claim 1, wherein the shipping box has an open top and wherein the at least one robot includes a first robot that is adapted to remove the at least one medication from the shipping box through the open top.

3. The temperature-controlled medication packaging system as set forth in claim 1, wherein the storage that includes the shipping box is held at a temperature that is no greater than forty degrees Fahrenheit.

4. The temperature-controlled medication packaging system as set forth in claim 3, wherein the storage has a curtain, and wherein the at least one robot includes a first robot that can penetrate through the curtain when retrieving the at least one medication directly from the shipping box.

5. The temperature-controlled medication packaging system as set forth in claim 4, wherein the storage includes a plurality of shipping boxes that are disposed on at least one platform, and wherein the plurality of shipping boxes have open tops.

6. The temperature-controlled medication packaging system as set forth in claim 5, wherein the at least one platform includes a plurality of platforms that contain different types of medications, and wherein all of the plurality of platforms are within a reach of the first robot.

7. The temperature-controlled medication packaging system as set forth in claim 1, wherein the shipping container is an insulated bag that acts as a moisture barrier between an environment outside the shipping container and an inside of the shipping container that can contain the at least one medication and the at least one coolant.

8. The temperature-controlled medication packaging system as set forth in claim 1, wherein the at least one robot includes a first robot that is adapted to retrieve the at least one medication directly from the shipping box and deliver the at least one medication to the shipping container in the package and includes a second robot that is adapted to retrieve and deliver to the shipping container at least one the at least one coolant and a filler and a temperature sensor and paperwork.

9. A method of filling a shipping container with a medication, containing the steps of:
picking at least one medication directly from a shipping box that is located within an environmentally controlled storage with at least one robot;
delivering the at least one medication to a packager that includes at least one shipping container with at least one robot; and
picking at least one coolant from a coolant supply and delivering the at least one coolant to the packager with the at least one robot.

10. The method as set forth in claim 9, further including the step of picking and delivering at least one of a filler, a temperature sensor, and paperwork to the packager with the at least one robot.

11. The method as set forth in claim 10, wherein the at least one robot includes a first robot and a second robot, and wherein the first robot performs the steps of picking the at least one medication from the shipping container and delivering the at least one medication to the packager, and
wherein the second robot performs the steps of picking and delivering the at least one of the filler, the temperature sensor, and the paperwork to the packager.

12. The method as set forth in claim 9, wherein the shipping box has an open top, and wherein the at least one robot picks the at least one medication from the shipping box through the open top.

13. The method as set forth in claim 9, further including the step of maintaining the environmentally controlled storage at a temperature that is no greater than forty degrees Fahrenheit.

14. The method as set forth in claim 9, wherein the environmentally controlled storage includes a curtain, and
wherein during the step of picking the at least one medication directly from the shipping box, the at least one robot penetrates through the curtain.

15. The method as set forth in claim 9, wherein the environmentally controlled storage includes a plurality of shipping boxes that are disposed on at least one platform, and wherein the plurality of shipping boxes have open tops.

16. The method as set forth in claim 15, wherein the at least one platform includes a plurality of platforms that contain different types of medications, and wherein all of the plurality of platforms are within a reach of the at least one robot.

17. The method as set forth in claim 9, wherein the shipping container is an insulated bag that acts as a moisture barrier between an environment outside the shipping container and an inside of the shipping container that can contain the at least one medication and the at least one coolant.

18. A temperature-controlled medication packaging system, comprising:
an environmentally controlled storage including a temperature-controlled interior containing a plurality of medications that are disposed within a plurality of shipping boxes, the shipping boxes having openings;
a coolant supply including at least one coolant;
a filler supply including a filler material;
a paperwork producer including paperwork related to the medications;
a packager including at least one insulated shipping container;
a first robotic arm that has a range of motion that includes at least the coolant supply and the packager;
a second robotic arm that has a range of motion that includes the packager and also includes at least one of the coolant supply, the filler supply, and the paperwork producer;
a controller in electrical communication with the first and second robotic arms; and
the controller being configured to operate the first robotic arm to retrieve at least one medication of the plurality of medications directly from one of the shipping boxes and deliver the at least one medication to the packager and being configured to operate the second robotic arm to retrieve and deliver to the packager at least one of the at least one coolant, the filler material, and the paperwork.

19. The temperature-controlled medication packaging system as set forth in claim 18, wherein the plurality of shipping boxes in the environmentally controlled storage are disposed on at least one platform, and wherein the plurality of shipping boxes have open tops.

20. The temperature-controlled medication packaging system as set forth in claim 19, wherein the at least one platform includes a plurality of platforms that support shipping boxes that contain different types of medications.
